SEQUENCE LISTING (25 sequences)

SEQ ID NO: 1 - protein, 239 aa, Escherichia coli
MVIKAQSPAGFAEEYIIESIWNNRFPPGTILPAERELSELIGVTRTTLREVLQRLARDGWLTIQHGKPTKVNNFWETSGLNILETLARLDHESVPQLIDNLLSVRTNISTIFIRTAFRQHPDKAQEVLATANEVADHADAFAELDYNIFRGLAFASGNPIYGLILNGMKGLYTRIGRHYFANPEARSLALGFYHKLSALCSEGAHDQVYETVRRYGHESGEIWHRMQKNLPGDLAIQGR

SEQ ID NO: 2 - DNA, 17 bp, Escherichia coli
  misc_feature (1)..(1): n is a, c, g, or t
nrctggtmygayswnwn

SEQ ID NO: 3 - DNA, 17 bp, Escherichia coli
atctggtacgaccagat

SEQ ID NO: 4 - DNA, 81 bp, Escherichia coli
atcggcatttctttaatcttttgtttgcatatttttaacacaaaatacacacttcgactcatctggtacgaccagatcacc

SEQ ID NO: 5 - DNA, 72 bp, Escherichia coli
atctggtacgaccagatttgacaatctggtacgaccagatgatactgagcacatcagcaggacgcactgacc

SEQ ID NO: 6 - DNA, 76 bp, Escherichia coli
aaaatttatcaaaaagagtgttgactatctggtacgaccagatgatacttagattcatctggtacgaccagatacc

SEQ ID NO: 7 - DNA, 80 bp, Escherichia coli
atcgtttaggcaccccaggctttacactttatgcttccggctcgtataatgtgtggaattgtgagcggataacaatttca

SEQ ID NO: 8 - DNA, 79 bp, Escherichia coli
atcgtttaggcaccccaggctttacaatctggtacgaccagattataatgtgtggaattgtgagcggataacaatttca

SEQ ID NO: 9 - DNA, 76 bp, Escherichia coli
atctggtacgaccagattttacaatctggtacgaccagattataatgtgtggaattgtgagcggataacaatttca

SEQ ID NO: 10 - DNA, 75 bp, Escherichia coli
aattgtgagcggataacaattgactatctggtacgaccagatgatacttagattcatctggtacgaccagatacc

SEQ ID NO: 11 - DNA, 77 bp, Escherichia coli
cactcattaggcaccccaggcctgatggctagctcagtcctagggactgtgctagcaggtcgacggtatcgataagc

SEQ ID NO: 12 - DNA, 77 bp, Escherichia coli
cactcattaggcaccccaggcctgatagctagctcagtcctagggattatgctagcaggtcgacggtatcgataagc

SEQ ID NO: 13 - DNA, 77 bp, Escherichia coli
cactcattaggcaccccaggcttgacagctagctcagtcctagggattgtgctagcaggtcgacggtatcgataagc

SEQ ID NO: 14 - DNA, 77 bp, Escherichia coli
cactcattaggcaccccaggctttacggctagctcagtcctaggtactatgctagcaggtcgacggtatcgataagc

SEQ ID NO: 15 - DNA, 77 bp, Escherichia coli
cactcattaggcaccccaggctttacggctagctcagtcctaggtatagtgctagcaggtcgacggtatcgataagc

SEQ ID NO: 16 - DNA, 77 bp, Escherichia coli
cactcattaggcaccccaggcttgacggctagctcagtcctaggtacagtgctagcaggtcgacggtatcgataagc

SEQ ID NO: 17 - DNA, 78 bp, Escherichia coli
atcgtttaggcaccccaggcctgatggctagctcagtcctagggactgtgctagcaattgtgagcggataacaattta

SEQ ID NO: 18 - DNA, 78 bp, Escherichia coli
atcgtttaggcaccccaggcctgatagctagctcagtcctagggattatgctagcaattgtgagcggataacaattta

SEQ ID NO: 19 - DNA, 78 bp, Escherichia coli
atcgtttaggcaccccaggcttgacagctagctcagtcctagggattgtgctagcaattgtgagcggataacaattta

SEQ ID NO: 20 - DNA, 78 bp, Escherichia coli
atcgtttaggcaccccaggctttacggctagctcagtcctaggtactatgctagcaattgtgagcggataacaattta

SEQ ID NO: 21 - DNA, 78 bp, Escherichia coli
atcgtttaggcaccccaggctttacggctagctcagtcctaggtatagtgctagcaattgtgagcggataacaattta

SEQ ID NO: 22 - DNA, 78 bp, Escherichia coli
atcgtttaggcaccccaggcttgacggctagctcagtcctaggtacagtgctagcaattgtgagcggataacaattta

SEQ ID NO: 23 - DNA, 71 bp, Escherichia coli
tttttgtctgctatcagcgtagttagccctctggtatgatgagtccaactttgttttgctgtgttatggaa

SEQ ID NO: 24 - DNA, 17 bp, Escherichia coli
atctggtacgaccagat

SEQ ID NO: 25 - DNA, 17 bp, Escherichia coli
  misc_feature (1)..(1): n is a, c, g, or t
nrctggtmygayswnwn

(19) United States
(12) Patent Application Publication
(10) Pub. No.: US 2013/0059295 A1
(43) Pub. Date: Mar. 7, 2013

(54) Host Cells and Methods for Producing Fatty Acid

(71) Applicants: Fuzhong Zhang, Ontario (CA); Eric J. Steen, San Francisco, CA (US); Jay D. Keasling, Berkeley, CA (US)

(72) Inventors: Fuzhong Zhang, Ontario (CA); Eric J. Steen, San Francisco, CA (US); Jay D. Keasling, Berkeley, CA (US)

(73) Assignee: THE REGENTS OF THE UNIVERSITY OF CALIFORNIA, Oakland, CA (US)

(21) Appl. No.: 13/549,034

(22) Filed: Jul. 13, 2012

Related U.S. Application Data
(60) Provisional application No. 61/507,994, filed on Jul. 14, 2011.

(51) Int. Cl. C12N 1/21; G01N 21/64; C12P 7/64

(52) U.S. Cl. 435/6.1; 435/252.33; 435/252.34; 435/252.3; 435/134

(57) ABSTRACT

The present invention provides for a genetically modified host cell capable of producing fatty acid comprising an increased expression of FadR, or a functional variant thereof. The host cell under environmental conditions wherein fatty acid is produced expresses an increased amount of FadR when compared to an unmodified host cell. The present invention also provides for a method of producing a fatty acid or FAAE in the host cell. The present invention provides for a genetically modified host cell comprising a fatty acid biosensor and one or more fatty acid-responsive promoter operably linked to one or more genes of interest that is heterologous to the fatty acid-responsive promoter.

### RELATED PATENT APPLICATIONS

The application claims priority to U.S. Provisional Patent Application Ser. No. 61/507,994, filed Jul. 14, 2011, which is herein incorporated by reference in its entirety.

### STATEMENT OF GOVERNMENTAL SUPPORT

The invention described and claimed herein was made utilizing funds supplied by the U.S. Department of Energy under Contract No. DE-AC02-05CH11231. The government has certain rights in this invention.

### FIELD OF THE INVENTION

The present invention is in the field of production of fatty acids, and in particular host cells that are genetically modified to produce fatty acids.

### BACKGROUND OF THE INVENTION

Fatty acids are important precursors that can be readily derived to produce biofuels, therapeutic compounds and expensive oils. It has been previously demonstrated that fatty acids can be produced from simple carbon source by microbes but with limited conversion yield. The low conversion yield resulted in high production costs of fatty acids and their derivatives. Currently, this problem is addressed mainly by engineering thioesterase, which is responsible for converting fatty acyl-CoA and fatty acyl-acyl carrier protein (ACP) into fatty acids.

### SUMMARY OF THE INVENTION

The present invention provides for a genetically modified host cell capable of producing fatty acid comprising an increased expression of FadR, or a functional variant thereof. The host cell under environmental conditions wherein fatty acid is produced expresses an increased amount of FadR when compared to an unmodified host cell.

The present invention also provides for a method of producing a fatty acid or fatty acid alkyl ester (FAAE) in a genetically modified host cell of the present invention. The method comprises culturing the genetically modified host cell of the present invention in a medium under a suitable condition such that the culturing results in the genetically modified host cell producing the fatty acid or FAAE, and optionally recovering the fatty acid or FAAE from the medium, wherein the recovering step is concurrent or subsequent to the culturing step. In some embodiments of the invention, the host cell comprises FadR, or a functional variant thereof, operably linked to an inducible promoter, and the method further comprises providing an inducer to the host cell, wherein the inducer increases expression from the inducible promoter. In some embodiments of the invention, the host cell is in a medium, and providing step comprises adding or introducing the inducer to the medium.

The present invention provides for a genetically modified host cell comprising a fatty acid biosensor and one or more fatty acid-responsive promoter operably linked to one or more genes of interest that is heterologous to the fatty acid-responsive promoter, wherein the expression of the fatty acid biosensor in the host cell is increased compared to the host cell if not unmodified. In some embodiments, the fatty acid biosensor is not native to the modified host cell.

The fatty acid biosensor is capable of regulating expression of the fatty acid-responsive promoter in response to the presence of an acyl-CoA or one or more fatty acid. In some embodiments of the invention, the fatty acid biosensor is fatty acid-responsive transcription factor or regulator, such as FadR. The fatty acid-responsive transcription factor or regulator can be native or heterologous to the host cell. In some embodiments of the invention, the fatty acid-responsive transcription factor or regulator is expressed from a gene residing on the host cell chromosome or on a vector in the host cell. In some embodiments of the invention, the gene of interest is a reporter gene, or an enzyme. The host cell can be used for screening fatty acid producing strains.

The present invention provides for a genetically modified host cell comprising a fatty acid-responsive transcription factor, and a fatty acid-responsive promoter operatively linked to a reporter gene, wherein the fatty acid-responsive promoter is capable of expression of the reporter gene with an activated form of the fatty acid-responsive transcription factor. In some embodiments of the invention, the fatty acid-responsive transcription factor is FadR, or a functional variant thereof, and the fatty acid-responsive promoter comprises the nucleotide sequence NRCTGGTMYGAYSWNWN, wherein R=A or G, M=A or C, Y=C or T, S=G or C, W=A or T, and N=A, G, T or C (SEQ ID NO:2). In some embodiments of the invention, the fatty acid-responsive promoter comprises the nucleotide sequence ATCTGGTACGACCAGAT (SEQ ID NO:3). In some embodiments of the invention, the reporter gene encodes a red fluorescent protein (RFP) or a green fluorescent protein (GFP).

The present invention provides for a method for sensing acyl-CoA and/or one or more fatty acids, comprising: (a) providing a genetically modified host cell of the present invention, and (b) detecting the expression of the reporter gene. In some embodiments of the invention, the (b) detecting step comprises detecting the gene product of the reporter gene. In some embodiments of the invention, the gene product of the reporter gene increases or decreases the doubling time of the modified host cell. In some embodiments of the invention, the gene product of the reporter gene causes the modified host cell to become resistant or sensitive to a compound.

The present invention provides for a method for screening or selecting a host cell that produces an acyl-CoA and/or one or more fatty acids, comprising: (a) providing a modified host cell of the present invention, (b) culturing the host cell, and (c) screening or selecting the host cell based the expression of the reporter gene by the host cell.

The fatty acid or FAAE produced using the host cell and/or method of the present invention can be useful for, or for conversion into, biofuels, fatty acid based oils, and/or therapeutic compounds.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows the biosynthetic pathway for the production of fatty acid ethyl ester (FAEE) in E. coli. The whole pathway was divided into three segments. Segment A contains the E. coli native fatty acid synthase and a cytoplasmic thioesterase gene (tesA), producing fatty acids. Segment B contains a pyruvate decarboxylase gene (pdc) and an alcohol dehydrogenase gene (adhB), producing ethanol. Segment C contains an acyl-CoA synthase gene (fadD) and a wax-ester synthase gene (atfA), producing FAEE as the end product.

FIG. 2 shows the design of Fatty acid biosensors. (a) Construct of fatty acid biosensor. In the absence of fatty acid, FadR binds to the FadR-regulatory promoter, P_{FadR}, represses the transcription of rfp. When there is fatty acid in presence, fatty acid is activated to acyl-CoA, which antagonizes the DNA binding activity of FadR, rfp transcription turns on. Fatty acid can be either added externally or produced intracellularly. (b) The DNA sequences of promoters used as fatty acid biosensor and compared with the native P_{lacUV5}. The bold sequences represent −10 and −35 region. FadR-binding sequence is colored blue. Transcript start sites are colored red. The nucleotide sequences comprising P_{fadBA}, P_{LR}, and P_{AR}, depicted in (b), are designated SEQ ID NO:4-6, respectively. (c) FadR repression of fatty acid-regulatory promoters. E. coli cells were transformed with fatty acid biosensor plasmids in the absence (gray columns) or the presence (black columns) of plasmid FadR. Cell culture fluorescence was measured and normalized to OD. (d) Response of fatty acid biosensors to exogenous oleic acid. Fatty acid biosensor plasmids pAR-rfp (black circles) or pLR-rfp (blue squares) were transformed into DH1 fadE knockout strain (filled dots) or fadD knockout strain (empty dots). Varied amount of oleic acid was added to the media and fluorescence was measured and normalized after incubation at 37° C. for 12 hours. (e) Response of fatty acid biosensors to internally produced fatty acids. Fatty acid biosensor plasmids were transformed into either wild-type DH1 or a fatty acid-producing strain (LTesA expressed). After incubation for three days, both fatty acid production (red dots) and cell culture fluorescence (black columns) were measured.

FIG. 3 shows inducible fatty acid-regulatory promoters. (a) Hybrid promoters created by the combination of P_{lacUV5} with P_{AR} and P_{LR}. The bold sequences represent −10 and −35 region. FadR-binding sequences are colored blue. LacI-binding sequences are colored brown. Transcript start sites are colored red. The nucleotide sequences comprising P_{lacUV5}, P_{FL1}, P_{FL2}, and P_{FL3}, depicted in (a), are designated SEQ ID NO:7-10, respectively. (b, c) Broad host range origin (BBR1) plasmids containing rfp gene under the control of hybrid promoters ( represents P_{FL1}, ▪ represents P_{FL2}, ▴ represents P_{FL3}, + represents P_{lacUV5}) were transformed into fadE knockout E. coli cells. Varied amount of inducers were added to the media and cell culture fluorescence were measured after 12 hours. Oleic acid concentrations were increased from 0.1 μM to 1 mM, followed by increasing IPTG concentration in the presence of 1 mM oleic acid (b). Alternatively, IPTG concentrations were increased from 0.1 μM to 1 mM, followed by increasing oleic acid concentration in the presence of 1 mM IPTG (c).

FIG. 4 shows the regulation of FAEE production by the sensory-regulatory system. (a) Sensory-regulatory network. Before the accumulation of fatty acids, FadR represses the fatty acid-regulatory promoters and inhibit the biosynthesis of ethanol and acyl-CoA. Production of fatty acids releases FadR from its DNA binding sites, simultaneously activates the biosynthesis of ethanol and acyl-CoA and the expression of was-ester synthase, which converts ethanol and acyl-CoA to FAEE. (b) Gene stability of FAEE-producing strains. FAEE-producing strains were incubated at 37° C. for three days for FAEE production. Plasmids were then prepared, restriction digested and analyzed by a 1% agarose gel. The three red arrows on the left indicate the expected size of integral plasmids, from top to bottom, they are plasmids containing segment B (expected 15-20 copies), segment C (expected 40-48 copies), and segment A (expected 10-15 copies). (c) FAEE production yields measured by GC-FID. FAEE-producing strains were induced with 1 mM IPTG and incubated at 37° C. for three days.

FIG. 5 shows dynamic regulation in comparison with static regulation. A series of constitutive promoters (a) or inducible promoters (c), were used to substitute either the P_{FL2} in segment B (b) or the P_{AR} in segment C (d) of the W strains. The bold sequences represent −10 and −35 region. LacI-binding sequences are colored brown. Transcript start sites are colored red. Gray columns represent fatty acids production levels and black columns represent FAEE production levels. The nucleotide sequences comprising P_{C2}, P_{C2}, P_{C3}, P_{C4}, P_{C5}, and P_{C6}, depicted in (a), are designated SEQ ID NO:11-16, respectively. The nucleotide sequences comprising P_{D1}, P_{D2}, P_{D3}, P_{D4}, P_{D5}, and P_{D6}, depicted in (c), are designated SEQ ID NO:17-22, respectively.

FIG. 6 shows the DNA sequence of E. coli chromosomal fadR promoter. The −35 and −10 regions are bold and underlined. Transcription start site is colored red. The chromosomal fadR promoter was aligned with the 17 bp FadR-binding sequence from fadBA promoter and the known FadR binding consensus (van Aalten, D. M., DiRusso, C. C. & Knudsen, J. The structural basis of acyl coenzyme A-dependent regulation of the transcription factor FadR. EMBO J 20, 2041-2050 (2001), hereby incorporated by reference). The identical sequence shared between fadR promoter and the 17 bp from fadBA promoter are highlighted in green. All the other nonidentical nucleotides are included in the consensus and highlighted yellow. The nucleotide sequences comprising the fadR promoter region, the 17 bp in the fadBA promoter region, and the FadR-binding consensus are designated SEQ ID NO:23-25, respectively.

FIG. 7 shows the fatty acid biosensor pLR-rfp turned fatty acid producing strains to a visible red color. Plasmid pLR-rfp were transformed into wild-type DH1 (A), fadE knockout DH1 (B) or fatty acid producing strains (cotransformed with a plasmid containing tesA gene) at either DH1 background (C) or fadE knockout DH1 (D).

FIG. 8 shows the time-course development of biosensor fluorescence in a fatty acid producing strain. The fatty acid producing contains a tesA gene under the control of a P_{lacUV5} promoter. This strain was transformed with pAR-rfp, its fluorescence was monitored (black circles) and compared with pAR-rfp transformed into E. coli DH1 cells (blue triangles). The fatty acid produced by the fatty acid producing strain was measured and presented by red squares.

FIG. 9 shows the ColE1 origin plasmids containing rfp gene under the control of a hybrid promoters ( represents P_{FL1}, ▪ represents P_{FL2}, ▴ represents P_{FL3}, + represents P_{lacUV5}) were transformed into fadE knockout E. coli cells. IPTG concentrations were increased from 0.1 μM to 1 mM, followed by increasing oleic acid concentration in the presence of 1 mM IPTG Inducers were added to the media and cell culture fluorescence were measured after 12 hours.

FIG. 10 shows the gene copy numbers after FAEE production. FAEE producing strains were incubated under production condition for three days. DNAs were isolated and qPCR was used to quantify the copy number of fadD and compared to that in the A2A strain.

FIG. 11 shows metabolite analysis of FAEE-producing strains. Five strains using either P_{lacUV5} or the fatty acid-regulated promoters (strain A2A, H, I, X, and J using P_{lacUV5}, P_{AR}, P_{FL1}, P_{FL2}, and P_{FL3} respectively, see Table 1) to control the expression of genes in the ethanol pathway were cultivated for FAEE production. Cell cultures were collected and the amount of ethanol (a) and acetate (b) were analyzed by HPLC (Example 2).

FIG. 12 shows strain A2A comprising engineered pathways for production of fatty acid-derived molecules from hemicelluloses or glucose. Flux through the E. coli fatty acid pathway (black lines) is increased to improve production of free fatty acids and acyl-CoAs by eliminating β-oxidation (knockouts are fadE), by overexpressing thioesterases (TES) and acyl-CoA ligases (ACL). Various products are produced from non-native pathways (orange lines) including biodiesel, alcohols and wax esters. Alcohols are produced directly from fatty acyl-CoAs by overexpressing fatty acyl-CoA reductases (FAR); the esters are produced by expressing an acyltransferase (AT) in conjunction with an alcohol-forming pathway; biodiesel is produced by introduction of an ethanol pathway (pdc and adhB) and wax esters were produced from the fatty alcohol pathway (FAR). Finally, expressing and secreting xylanases (xyn10B and xsa) allowed for the utilization of hemicellulose. Overexpressed genes or operons are indicated; green triangles represent the lacUV5 promoter. AcAld, acetaldehyde; EtOH, ethanol; pyr, pyruvate. FIG. 12 is taken from ref. 4 of Example 2.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The terms “host cell” and “host microorganism” are used interchangeably herein to refer to a living biological cell that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host microorganism, wherein the expression vector contains a nucleic acid sequence coding for an enzyme that is not normally found in a host microorganism. With reference to the host microorganism's genome, then, the nucleic acid sequence that codes for the enzyme is heterologous.

The terms “expression vector” or “vector” refer to a compound and/or composition that transduces, transforms, or infects a host microorganism, thereby causing the cell to express nucleic acids and/or proteins other than those native to the cell, or in a manner not native to the cell. An “expression vector” contains a sequence of nucleic acids (ordinarily RNA or DNA) to be expressed by the host microorganism. Optionally, the expression vector also comprises materials to aid in achieving entry of the nucleic acid into the host microorganism, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host microorganism and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host microorganism or cell. Only when the sequence of nucleic acids becomes stably replicated by the cell does the host microorganism or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; internucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

The term “functional variant” describes an enzyme that has a polypeptide sequence that is at least 70%, 75%, 80%, 85%, 90%, 95% or 99% identical to any one of the regulators or enzymes described herein. The “functional variant” regulator or enzyme may retain amino acids residues that are recognized as conserved for the enzyme, and may have non-conserved amino acid residues substituted or found to be of a different amino acid, or amino acid(s) inserted or deleted, but which does not affect or has insignificant effect its biological activity, such DNA-binding activity or enzymatic activity, as compared to the regulator or enzyme described herein. The “functional variant” regulator or enzyme has an biological activity that is identical or essentially identical to the biological activity of the regulator or enzyme described herein. The “functional variant” regulator or enzyme may be found in nature, i.e. naturally occurring, or be an engineered mutant thereof.

The term “reporter gene” means a gene whose phenotypic expression is easy to monitor or can be monitored, and which is linked to a promoter which is not the promoter of the gene itself in nature.

These and other objects, advantages, and features of the invention will become apparent to those persons skilled in the art upon reading the details of the invention as more fully described below.

FadR is a dual DNA-binding transcriptional regulator which is involved in several processes in the fatty acid pathway, including fatty acid activation, membrane transportation, degradation and conversion to unsaturated fatty acids. FadR controls the expression of several genes involved in fatty acid transport and β-oxidation (fadBA, fadD, fadL, and fadE). FadR's DNA-binding activity is regulated by FadR binding to acyl-CoA, the activated form of fatty acid and to a lesser extent fatty acid themselves. In the absence of fatty acid, FadR forms a homodimer and binds to specific DNA sequences of a promoter and controls the expression of several genes. When fatty acid is present, the fatty acid is activated by acyl-CoA synthase to acyl-CoA. Acyl-CoA then binds to FadR to trigger a conformation change on FadR and releases FadR from its cognate DNA sequence.

FadR activates the transcription of at least three genes required for unsaturated fatty acid biosynthesis (fabA, fabB, and iclR). It belongs to the GntR family of transcriptional regulators. FadR is a transcriptional factor that regulates several processes in fatty acid pathway. FadR down-regulates the fadD and fadL genes, whose gene products are responsible for fatty acid activation and membrane transportation; and several genes in fatty acid degradation pathway, including fadE, fadA, fadB and fadH. FadR up-regulates fabA and fabB. These gene products are involved in unsaturated fatty acid biosynthesis. FadR regulates these genes by binding to a specific DNA sequence in their promoter region. The native fadR gene is also self-regulated. This means that when there is enough FadR protein present, FadR binds to its own promoter and inhibits its gene expression.

Increasing the cellular FadR concentration lowers the fatty acid degradation rate and enhances unsaturated fatty acid biosynthesis, which results in increasing the total fatty acid production. In one embodiment of the invention, the host cell comprises a plasmid (such as pE8a-fadR) that contains an extra copy of fadR gene under the control of an inducible promoter. Expression of fadR gene from this plasmid is controlled by the inducer arabinose, but is not responsive to the cellular FadR concentration. Using a fatty acid production strain (E. coli ΔfadE strain with a thioesterase plasmid pA 10-LtesA), the total yield of fatty acid is increased by 3˜4 fold in the absence of arabinose. When the amount of the inducer is titrated, maximal production is observed at about 0.4% arabinose. Under this condition (minimal media with 2% glucose as carbon source in shaking test tubes), the total fatty acid production yield is about 6.0 g/L after three days incubation at 37° C. This yield is six times higher than previously reported results and corresponds to an about 80% conversion on carbon source.

The amino acid sequence of E. coli FadR is as follows:

(SEQ ID NO: 1)
MVIKAQSPAG FAEEYIIESI WNNRFPPGTI LPAERELSEL IGVTRTTLRE VLQRLARDGW
LTIQHGKPTK VNNFWETSGL NILETLARLD HESVPQLIDN LLSVRTNIST IFIRTAFRQH
PDKAQEVLAT ANEVADHADA FAELDYNIFR GLAFASGNPI YGLILNGMKG LYTRIGRHYF
ANPEARSLAL GFYHKLSALC SEGAHDQVYE TVRRYGHESG EIWHRMQKNL PGDLAIQGR

In some embodiments of the invention, the host cell comprises an open reading frame (ORF) encoding a FadR, or a functional variant thereof, operably linked to a promoter heterologous to FadR. In some embodiments of the invention, the promoter is not regulated by the presence or concentration of FadR in the host cell. In some embodiments of the invention, the heterologous promoter is a constitutive or inducible promoter. In some embodiments of the invention, the inducible promoter can be any inducible promoter that increases or elevates expression when an inducer is present in the host cell or environment of the host cell. In some embodiments of the invention, the inducer can be introduced to the host cell by introducing the inducer to the environment of the host cell, i.e. the inducer can enter into the host cell. In some embodiments of the invention, the ORF is operably linked to an inducible promoter, and one skilled in the art is capable of adjusting the amount of inducer present in order to determine the amount of inducer in the environment of the cell in order to obtain the optimum or maximum production of fatty acid.

In some embodiments of the invention, the host cell comprises a plurality of the ORF encoding a FadR, or a functional variant thereof. The ORFs of the plurality of ORF can each independently have a nucleotide sequence different from another ORF. For example, every ORF within the host cell can have a different nucleotide sequence and/or encode a FadR, or a functional variant thereof, with a different amino acid sequence, or every ORF with the host cell can have a different nucleotide sequence and each ORF encodes a FadR, or a functional variant thereof, with the same amino acid sequence, or every ORF with the host cell can have the same nucleotide sequence. In some embodiments of the invention, an ORF encoding a FadR, or a functional variant thereof, can be optimized for expression of that particular amino acid sequence. In some embodiments of the invention, an ORF has a naturally occurring nucleotide sequence. In some embodiments of the invention, an ORF encodes a FadR with a naturally occurring amino acid sequence.

In some embodiments of the invention, the host cell comprises one or more ORFs encoding proteins, or functional variants thereof, involved in the activation or transportation of fatty acid, such as TesA, FabA, FabB, FabD, and FabL. In some embodiments of the invention, the host cell comprises one or more ORFs encoding proteins, or functional variants thereof, involved in the unsaturated fatty acid biosynthesis, such as FabA and FabB. In some embodiments of the invention, the host cell comprises the genes for fatty acid production native to the host cell.

An ORF can stably reside on the chromosome of the host cell. An ORF can reside on a vector. The vector can be capable of stable maintenance with the host cell. The host cell can comprise one or more ORFs residing on the chromosome of the host cell, one or more vectors comprising one or more ORFs, or both.

In some embodiments of the invention, the host cell is knocked out for the expression of FadR from the chromosome. U.S. Patent Application Pub. No. 2004/0132145 discloses a method of constructing a FadR knock-out microorganism. In some embodiments of the invention, the host cell is knocked out for the expression of FadE from the chromosome.

In some embodiments of the invention, the host cell is capable of producing equal to or more than about 1.0, 2.0, 3.0, 4.0, 5.0, or 6.0 g/L of fatty acid. In some embodiments of the invention, the host cell is capable of producing from about 1.0, 2.0, or 3.0 g/L to about 4.0, 5.0, or 6.0 g/L of fatty acid. In some embodiments of the invention, the yield of fatty acid is under conditions comprising growth in a minimal media comprising 2% glucose and three days of incubation at 37° C.

In some embodiments of the invention, the host cell is capable of producing fatty acid from a conversion equal to or more than about 10, 20, 30, 40, 50, 60, 70, or 80% of the carbon source provided to the host cell. In some embodiments of the invention, the host cell is capable of producing fatty acid from a conversion ranging from about 10, 20, 30, or 40% to about 50, 60, 70, or 80% of the carbon source provided to the host cell. In some embodiments of the invention, the percent conversion to a fatty acid from the carbon source provided to the host cell is under conditions comprising growth in a minimal media comprising 2% glucose and three days of incubation at 37° C.

In some embodiments of the invention, the host cell further comprises one or more enzymes, or a functional variant thereof, capable of capable of producing a fatty acid alkyl ester (FAAE) from the fatty acid and an alkyl alcohol. In some embodiments of the invention, the fatty acid alkyl ester (FAAE) is a fatty acid ethyl ester (FAEE) and the alkyl alcohol is ethanol. Such suitable enzymes are taught in U.S. 2010/0180491 and WO 2009/006386, both of which are hereby incorporated by reference.

The present invention also provides for a method of producing a fatty acid or FAAE in a genetically modified host cell of the present invention. The method comprises culturing the genetically modified host cell of the present invention in a medium under a suitable condition such that the culturing results in the genetically modified host cell producing the fatty acid or FAAE, and optionally recovering the fatty acid or FAAE from the medium, wherein the recovering step is concurrent or subsequent to the culturing step. In some embodiments of the invention, the host cell comprises FadR, or a functional variant thereof, operably linked to an inducible promoter, and the method further comprises providing an inducer to the host cell, wherein the inducer increases expression from the inducible promoter. In some embodiments of the invention, the host cell is in a medium, and providing step comprises adding or introducing the inducer to the medium.

The present invention provides for a method for screening or selecting a host cell that produces an acyl-CoA and/or one or more fatty acids, comprising: (a) providing a modified host cell of the present invention, (b) culturing the host cell, and (c) screening or selecting the host cell based the expression of the reporter gene by the host cell.

In some embodiments of the present invention, the method for screening or selecting a host cell that produces an acyl-CoA and/or one or more fatty acids, comprises: (a) providing a plurality of modified host cells of the present invention wherein the modified host cells of different modification are in separate cultures, (b) culturing each separate culture of host cell, (c) screening or selecting the host cell based the expression of the reporter gene by the host cell, and (d) comparing the expression of the reporter genes of the separate cultures. In some embodiments of the present invention, the (d) comprising step comprises identifying one or more cultures, and/or the corresponding host cell, that have an increased expression of the gene product of the reporter gene.

In some embodiments, the method is a method for selecting a host cell that produces an acyl-CoA and/or one or more fatty acids, wherein the selection is a positive selection or a negative selection. When the selection is positive selection, the selecting step selects for host cells that have a higher expression of a reporter gene that increases the probability of remaining viable and doubling, and thus have a higher probability of remaining viable and doubling. When the selection is negative selection, the selecting step selects for host cells that have a lower expression of the reporter gene that decreases the probability of remaining viable and doubling, and thus have a higher probability of remaining viable and doubling.

In one embodiment of the present invention, the method for selecting an E. coli host cell that produces an acyl-CoA and/or one or more fatty acids comprises: (a) providing a plurality of modified E. coli host cells of the present invention wherein the modified host cells of different modification are in separate cultures, (b) culturing each separate culture of host cell, (c) selecting the host cell based the expression of the reporter gene by the host cell, and (d) comparing the expression of the reporter genes of the separate cultures, wherein the selecting is a positive selecting.

In another embodiment of the present invention, the method for selecting an E. coli host cell that produces an acyl-CoA and/or one or more fatty acids comprises: (a) providing a plurality of modified E. coli host cells of the present invention wherein the modified host cells of different modification are in separate cultures, (b) culturing each separate culture of host cell, (c) selecting the host cell based the expression of the reporter gene by the host cell, and (d) comparing the expression of the reporter genes of the separate cultures, wherein the selecting is a negative selecting.

In some embodiments of the invention, the compound is an antibiotic and the reporter gene is an antibiotic resistance gene which confers resistance to the antibiotic. In some embodiments of the invention, the reporter gene is cat or bla. The reporter gene can be used as a positive selection or as a negative selection. Positive selection occurs when the increased expression of the gene product of the reporter gene increases the probability that the host cell would remain viable and complete doubling. Examples of reporter genes that confer positive selection are antibiotic resistance genes that confer resistance to an antibiotic of the host cell when the host cell is cultured or grown in a culture containing the antibiotic. An example of such as is a β-lactamase, encoded by the bla gene. Other examples of reporter genes that confer positive selection are genes encoding enzymes that are required by the host cell to metabolize a specific nutrient source which is required by the host cell in order to remain viable and double. Negative selection occurs when the increased expression of the gene product of the reporter gene decreases the probability that the host cell would remain viable and complete doubling. Examples of reporter genes that confer negative selection are genes which when expressed inhibit resistance to an antibiotic of the host cell when the host cell is cultured or grown in a culture containing the antibiotic. An example of such as inhibitor is a β-lactamase inhibitor, such as clavulanic acid, which inhibits a β-lactamase, such as ampicillin.

The biosensor can be applied to other suitable ligand-responsive repressors that operate in a similar manner to FadR except in response to different ligands. Such suitable ligand-responsive repressors are indicated in Table 2. The amino acid sequence of these ligand-responsive repressors and their corresponding DNA sequences to which each binds are well known.

TABLE 2
Ligand-responsive Repressors.

| Protein | Organism | Ligands | K_{d} (apparent) | Ref. |
|---|---|---|---|---|
| EnrR | Escherichia coli | Nalidixic acid, salicylate, caronyl cyanide | 1.3-11.1 μM | Lomovskaya et al., |
| | | m-chlorophenylhydrazone, | | 1995 |
| | | 2,4-dinitrophenol, ethidium bromide | | Brooun et al., 1999 |
| | | | | Xiong, et al., 2000 |
| BadR | Rhodopseudomonas palustris | Benzoate, 4-hydroxybenzoate | — | Eglard and Harwood, |
| | | | | 1999 |
| CoaR | Comamonas testosteroni | 3-chlorobenzoate, protocatechuate | — | Providentl and |
| | | | | Wyncham, 2001 |
| CinR | Butyrivibrio fibrisolvens | Cinnamic acid sugar esters | — | Dalrymple and |
| | | | | Swadling, 1997 |
| HcaR | Acinetobacter sp. strain ADP1 | hydroxycinnamoyl-CoA thioesters | — | Parke and Ornston, |
| | | | | 2003 |
| HcaR | Escherichia coli | 4-hydroxyphenylacetic acid, 3-hydroxy- | — | Galan et al., 2003 |
| | | phenylacetic acid, 3,4-hydroxyphenyl- | | |
| | | acetic acid | | |
| MarR | Escherichia coli | salicylate, plumbagin, 2,4-dinitrophenol, | 0.5-1 mM | Cohen et al., |
| | | menadione | | 1993b |
| | | | | Seoane and Levy, |
| | | | | 1995 |
| | | | | Martin and Rosner, |
| | | | | 1995 |
| | | | | Alekshun and |
| | | | | Levy, 1999a |
| | | | | Alekshun et al., |
| | | | | 2001 |
| ChrR | Xantnomonas campestris and | tert-butyl hydroperoxide, cumene | — | Sukchawalit et al., |
| | Bacillus subtilis | hydroperoxide | | 2001 |
| | | | | Panmanae et al., |
| | | | | 2002 |
| | | | | Fuangthong et al., |
| | | | | 2001 |
| | | | | Fuangthong and |
| | | | | Helmann, 2002 |
| HucR | Dainococcus radiodurans | uric acid, salicylate | 11.6 μM | Wilkinson and |
| | | | | Grove, 2004 |
| | | | | Wilkinson and |
| | | | | Grove, 2005 |

Each of the reference cited is hereby incorporated by reference as though each is individually and separately incorporated by reference.

For example, in one embodiment of the invention, the genetically modified host cell which expresses Rhodopseudomonas pelustris BadR, or a functional variant thereof, comprising using a benzoate or 4-hydroxybenzoate biosensor to regulate benzoate or 4-hydroxybenzoate-responsive promoters operably linked to one or more genes of interest that is heterologous to the benzoate or 4-hydroxybenzoate-responsive promoter. This system can be applied to each ligand-responsive repressor listed in Table 2.

The fatty acid or FAAE produced using the host cell and/or method of the present invention can be useful for, or for conversion into, biofuels, fatty acid based oils, and/or therapeutic compounds.

The nucleic acid constructs of the present invention comprise nucleic acid sequences encoding one or more of the subject regulator or enzyme. The nucleic acid of the subject enzymes are operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject regulator or enzyme are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, XhoI, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host microorganism simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (Lad repressor protein changes conformation when contacted with lactose, thereby preventing the Lad repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host microorganism. Also, microinjection of the nucleic acid sequencers) provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

The host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of aromatic amino acid ensured. When added, the intermediate is present in an excess amount in the culture medium.

As the host cell grows and/or multiplies, expression of the regulators or enzymes for producing the fatty acids is effected. Once expressed, the enzymes catalyze the steps necessary for carrying out the steps of fatty acid and/or FAAE production. If an intermediate has been introduced, the expressed enzymes catalyze those steps necessary to convert the intermediate into the respective oxidation product. Any means for recovering the oxidation product from the host cell may be used. For example, the host cell may be harvested and subjected to hypotonic conditions, thereby lysing the cells. The lysate may then be centrifuged and the supernatant subjected to high performance liquid chromatography (HPLC) or gas chromatography (GC). Once the fatty acid or FAEE is recovered, modification, as desired, may be carried out on the fatty acid or FAEE.

### Host Cells

The host cells of the present invention are genetically modified in that heterologous nucleic acid have been introduced into the host cells, and as such the genetically modified host cells do not occur in nature. The suitable host cell is one capable of expressing a nucleic acid construct encoding one or more regulators or enzymes described herein. The gene(s) encoding the regulator(s) or enzymes (s) may be heterologous to the host cell or the gene may be native to the host cell but is operatively linked to a heterologous promoter and one or more control regions which result in a higher expression of the gene in the host cell.

The regulators or enzymes can be native or heterologous to the host cell. Where the enzyme is native to the host cell, the host cell is genetically modified to modulate expression of the regulators or enzymes. This modification can involve the modification of the chromosomal gene encoding the regulators or enzymes in the host cell or a nucleic acid construct encoding the gene of the regulators or enzymes is introduced into the host cell. One of the effects of the modification is the expression of the regulators or enzymes is modulated in the host cell, such as the increased expression of the regulators or enzymes in the host cell as compared to the expression of the enzyme in an unmodified host cell.

In some embodiments of the invention, the host cell is a microorganism from the Enterobacteriaceae family. In some embodiments of the invention, the host cell is a Gram negative bacterium. In some embodiments of the invention, the host cell is a microorganism from the Escherichia, Salmonella, Vibrio, Pasteurella, Haemophilus, or Pseudomonas genus. In some embodiments of the invention, the host cell is a microorganism from the species Escherichia coli, Salmonella enterica, Vibrio cholerae, Pasteurella multocida, Haemophilus influenza, or Pseudomonas aeruginosa.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Increasing fadR Expression to Increase Fatty Acid Production

Increasing the cellular FadR concentration lowers the fatty acid degradation rate and enhances unsaturated fatty acid biosynthesis, which results in increasing the total fatty acid production. In one embodiment of the invention, the host cell comprises a plasmid (such as pE8a-fadR) that contains an extra copy of fadR gene under the control of an inducible promoter. Expression of fadR gene from this plasmid is controlled by the inducer arabinose, but is not responsive to the cellular FadR concentration. Using a fatty acid production strain (E. coli ΔfadE strain with a thioesterase plasmid pA 10-LtesA), the total yield of fatty acid is increased by 3˜4 fold in the absence of arabinose. When the amount of the inducer is titrated, maximal production is observed at about 0.4% arabinose. Under this condition (minimal media with 2% glucose as carbon source in shaking test tubes), the total fatty acid production yield is about 6.0 g/L after three days incubation at 37° C. This yield is six times higher than previously reported results and corresponds to an about 80% conversion on carbon source.

The mechanism of FadR enhanced fatty acid production is studied. Using RNA microarray, E. coli transcript levels are measured and compared between production strains with and without pE8a-fadR. The presence of pE8a-fadR caused transcript changes on a broad range of genes. More than 240 genes show up-regulation with more than 2-fold change and 99% confidence, and 255 genes showed down-regulation. For genes directed involved in fatty acid pathway, fabB showed the greatest change, 2.8-fold increasing on its transcript level. It is known that the fabB gene product catalyzes chain elongation reactions during fatty acid biosynthesis. To test the role of fabB played in fadR enhanced fatty acid production, the fadR gene in plasmid pE8a-fadR is replaced by the fabB gene to create plasmid pE8a-fabB. Fatty acid production strain (E. coli ΔfadE strain with plasmid pA 10-LtesA) carrying pE8a-fabB increases the yield by 2-fold at optimal inducer concentration. These results imply that FadR increases fatty acid production by causing a global change on the metabolism of the cell rather than acting on one specific gene.

### Example 2

### Design of Fatty Acid Biosensors and Sensory-Regulatory Devices for Biodiesel Production

### Introduction

With the development of metabolic engineering, microbial production of chemicals has demonstrated an attractive alternative to chemical synthesis. Many types of compounds have been biosynthesized, including pharmaceuticals^{1, 2}, fine chemicals³ (i.e., pigments, flavors, and vitamins), bulk chemicals (i.e., solvents and polymer precursors), and biofuels^{4, 5}. In these examples, heterologous genes or pathways were expressed in host microbial cells, and the engineered microbes converted simple sugars or degradable cellulosic biomass into target chemicals by a series of enzymatic reactions. For practical application, the engineered microbes need to produce target compounds in high product titers and conversion yields, which are extremely important for low value products, such as bulk chemicals and biofuels.

High product titers and yields are often limited by the imbalances in metabolism. Expression of pathway genes at too low a level is not adequate for chemical conversion. On the other side, expression at too high a level will divert cellular resources to the production of unnecessary RNAs, proteins, or intermediates, which consume large amounts of cellular resources. Furthermore, heterologous enzymes or pathway intermediates are sometimes toxic to the host. Over-production of toxic enzymes or intermediates leads to growth retardation or adaptive responses such as gene modification to remove or inactivate the pathway genes, causing reduced yield and productivity⁶. Several strategies have been developed to regulate gene expression levels including engineering the strengths of promoters⁷, intergenic regions⁸, and ribosome binding sites (RBSs)⁹. These methods provide static control of gene expression level, where gene expression levels are fixed without sensing changes in metabolic status or pathway output. Any deviation away the chosen condition may result in suboptimal productivity. Farmer and Liao presented the first example of dynamic regulation on heterologous pathways by using acetyl phosphate (ACP) as an indirect indicator for excess glucolytic flux to regulate the biosynthesis of lycopene¹⁰. Dynamic regulation allows a host strain to adapt its metabolic flux at real time, provides more reliability on metabolic balancing. Dynamic regulation is not limited to monitor glucose flux, but also environmental signals, cell growth, and more importantly, on the flux of the engineered pathways. Theoretically, a regulatory system that directly senses the concentration of critical pathway intermediates and dynamically regulates the expression of pathway genes will allow the delivery of intermediates to the proper level and optimize a heterologous pathway to its best productivity. Such technique will be especially useful for compounds that are produced from very long pathways or from the convergence of multiple pathway segments, where timing the expression of each pathway segment plays critical roles in productivity.

We aim to develop a sensory-regulatory system that dynamically senses the concentration of a pathway intermediate and regulates the expression of engineered pathways according to the flux of the intermediate. Engineering of a sensory-regulatory device requires three components: a cellular biosensor that real-time senses the cellular concentration of a pathway intermediate; a method to regulate the pathway flux; and a connection to transfer the sensor signal into the regulatory activity. Cellular biosensors can be developed from several sources including but not limited to the adaption of two-component system sensor domains, the use of ligand-responsive transcriptional factors, and computational designed artificial biosensor¹¹. Pathway regulation can be achieved transcriptionally by the engineering of promoters, translationally by the engineering of intergenic regions, RBSs or RNAs, and post-translationally by engineering of enzymes.

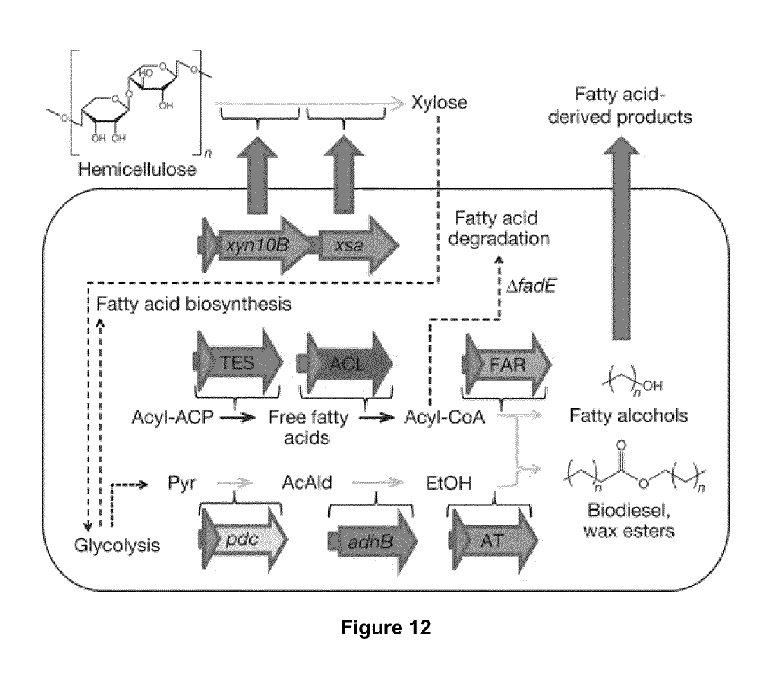

Here we focus on the engineered biodiesels biosynthetic pathway. Biodiesel, in the form of fatty acid ethyl ester (FAEE), is an excellent diesel fuel replacement due to its low water solubility, high energy density, and low toxicity to host cells¹². A FAEE biosynthetic E. coli strain, A2A, has been recently developed, which converted 2% glucose into FAEE with a 9.4% yield⁴ (see FIG. 12). For practical replacement of petroleum-derived diesel fuel with biodiesel, further improvements in productivity and conversion yield are required. However enhancing yield close to the theoretical maximum is extremely difficult, which requires perfect balancing in host metabolism.

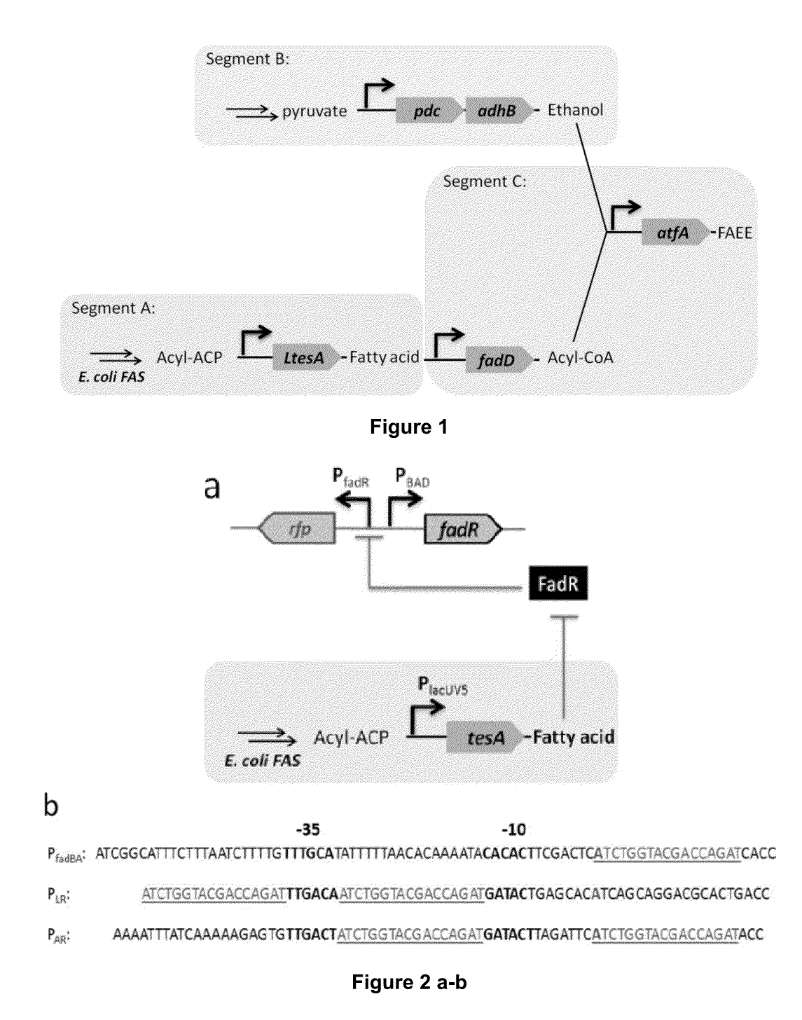

The previously developed FAEE biosynthetic pathway contains three segments⁴ (FIG. 1). Segment A uses the native E. coli fatty acid pathway and expresses a cytosolic thioesterase LTesA (coded by ltesA) to hydrolyze acyl-acyl carrier proteins (acyl-ACPs) and produce free fatty acids. Segment B contains an ethanol biosynthetic pathway which converts cellular pyruvate into ethanol. Segment C contains an acyl-CoA synthase (coded by fadD) and a wax-ester synthase (coded by atfA), whose enzyme products converge the products from the previous two segments by activating fatty acids to acyl-CoAs and esterifying acyl-CoAs and alcohols to FAEEs. Close examination of this engineered pathway will find: (i) ethanol is a toxic intermediate, both production level and the timing of ethanol production need to be regulated; (ii) activation of fatty acid to acyl-CoA is a reversible step because LTesA is able to hydrolyze acyl-CoA to fatty acid, early production of acyl-CoA is not necessary; (iii) Acyl-CoA is the precursor of the fatty acid β-oxidation pathway, which leads to the degradation of fatty acids. Acyl-CoA overproduction may lead to decrease in FAEE production yield. Ideally, both segment B and C are controlled according to the availability of fatty acid: acyl-CoA and ethanol are biosynthesized concurrently and produced only when there is sufficient fatty acid available, and they are converted to FAEE immediately after their biosynthesis. In order to achieve this goal, we designed biosensors to monitor the cellular concentration of fatty acids, and developed a sensory-regulation device to control FAEE pathway.

### Results

### Design of Fatty Acid Biosensors.

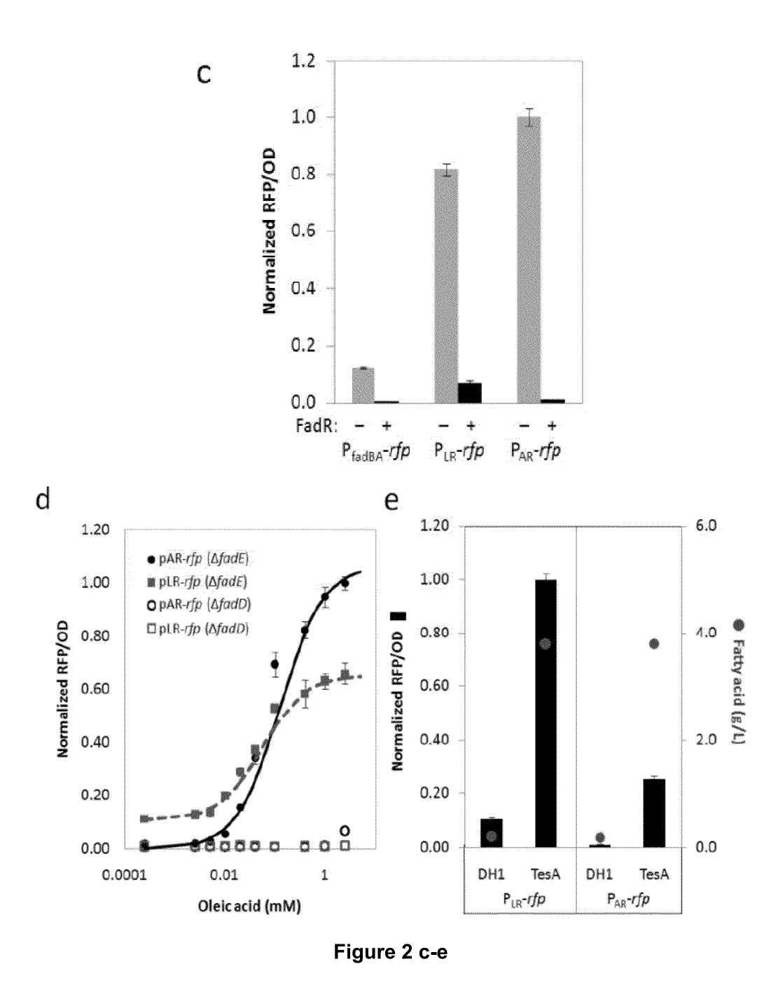

We designed fatty acid biosensors based on the E. coli transcriptional factor FadR. FadR is a global regulator that binds to specific DNA sequences and controls the expression of several genes, which involve in fatty acid biosynthesis, degradation, and membrane transportation¹³. The DNA binding activity of FadR is specifically antagonized by acyl-CoAs¹⁴, the activated form of fatty acids. Although previous results from electrophoretic mobility shift assay (EMSA) showed that free fatty acid can also eliminate the DNA binding activity of FadR, fatty acid were only effective at micromolar concentration range as compared to nanomolar for acyl-CoA¹⁵. Native FadR-regulatory promoters have limited output ranges: the E. coli fadBA promoter (P_{fadBA}) exhibited 5-fold increased expression level upon the addition of 5 mM oleic acid¹⁶, and the fabA promoter exhibited 2-10 fold changes depending on the acyl chain length¹⁷. In order to increase the output range, we designed two synthetic fatty acid-regulatory promoters, P_{LR} and P_{AR}, based on a phage lambda promoter P_{L} and a phage T7 promoter P_{A1} respectively¹⁸. In detail, the 17 bp FadR-binding DNA sequence from fadBA promoter (the strongest known binding site for FadR, K_{d}=0.2 nM¹⁹) was integrated into two locations of phage promoters flanking the −35 region in P_{LR} and −10 region in P_{AR} (FIG. 2b). Two synthetic fatty acid biosensor plasmids, pLR-rfp and pAR-rfp, were constructed by cloning a red fluorescence protein (rfp) gene under the control of P_{LR} and P_{AR} respectively (FIG. 2a). In the absence of fatty acid, FadR is expected to bind to the 17 bp DNA sequences, interferes with RNA polymerase from binding to the phage promoter, leading to the inhibition of rfp transcription. When fatty acid concentration increases, fatty acid is expected to be activated to acyl-CoA by acyl-CoA synthase. Acyl-CoA in turn binds to FadR and releases FadR from the synthetic promoter, initiating RFP transcription.

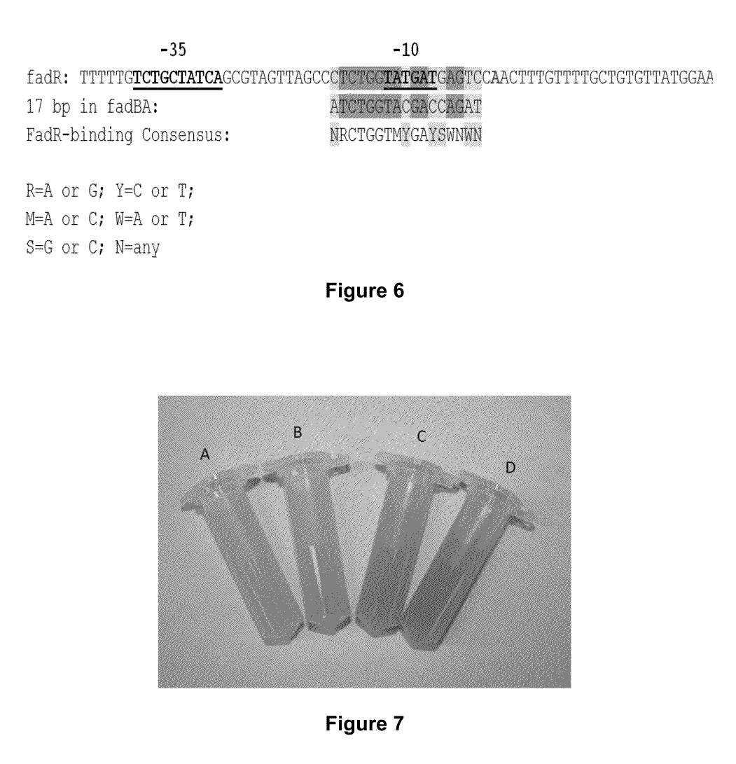

We first tested the response of the synthetic promoters towards FadR repression. The chromosomal fadR promoter contains a DNA sequence homologous to the 17 bp FadR-binding sequence¹⁹ (FIG. 6), which implies that the native FadR is tightly self-regulated. It is supported by the strong cellular fluorescence after transformation of fatty acid biosensor plasmids into E. coli cells (FIG. 2c). When a plasmid fadR under the control of a P_{BAD} promoter was expressed, which increased the fadR mRNA concentration by 7.5-fold (data not shown), cellular fluorescence was repressed significantly in all three constructs (FIG. 2c). As compared to the 22-fold repression on P_{fadBA}, P_{AR} showed 89-fold repression, more sensitive than the native promoter. Next, the responses of biosensors towards fatty acid were evaluated. Biosensor plasmids pLR-rfp and pAR-rfp were transformed into fadE knockout DH1 E. coli cells and oleic acid was exogenously added to the media. The enzyme product of fadE catalyzes the first step in fatty acid degradation. Deletion of fadE is expected to slow down the degradation of exogenous oleic acid and maintain the oleic acid concentration in the culture. E. coli transformed with either plasmid showed oleic acid dependent activation of fluorescence over a broad concentration range from 0.1 μM to the solubility limit of oleic acid in aqueous solution, 5 mM (FIG. 2d). In the case of pAR-rfp, a 60-fold fluorescence change was observed upon the addition of oleic acid, greater than all the reported native fatty acid-regulatory promoters. The apparent half maximal effective concentration (EC₅₀) of oleic acid is 35-60 much higher than the K_{d} of FadR binding to either oleoyl-CoA or oleic acid, indicating that only a small proportion of oleic acid was diffused into the cell. In fact, when acyl-CoA synthase gene (fadD) was knockout, no induction of RFP expression was detected up to the addition of 1 mM oleic acid (FIG. 2d), suggesting that with 1 mM oleic acid in the medium, its intracellular concentration was below 5 μM, the K_{d} of FadR binding to oleic acid¹⁵. The inability to activate RFP expression in the fadD knockout strain also proved that oleoyl-CoA, not oleic acid, induced the RFP expression in the fadE knockout strain.

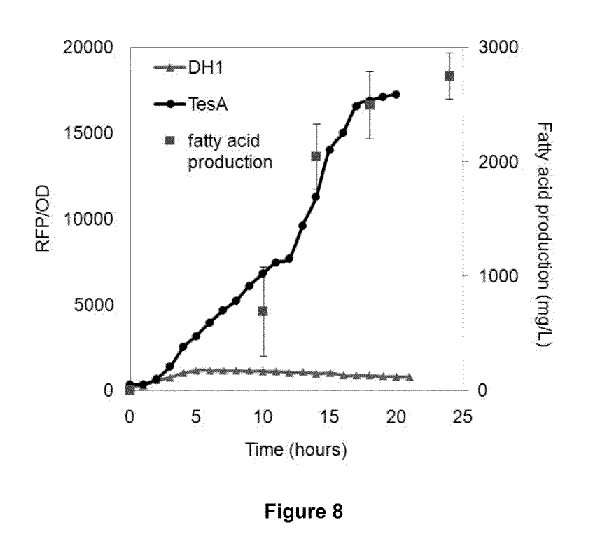

We next tested the response of fatty acid biosensors towards internally produced fatty acids. To do so, pLR-rfp and pAR-rfp were transformed into a fatty acid-producing strain. This strain contains tesA under the control of a P_{lacUV5} promoter (FIG. 2a) and produced 3.8 g/L fatty acid after incubation for three days. As compared to the wild-type DH1 cell, pLR-rfp and pAR-rfp in the fatty acid-producing strain exhibited 10-fold and 25-fold increasing in fluorescence intensity (FIG. 2d) and turned the cell culture to a visible red color (FIG. 7). The time-course of fluorescence development correlated well with the time-course of fatty acid production, confirming that the RFP expression was turned on by intracellular fatty acids (FIG. 8). Furthermore, the fatty acid-producing strain exhibited enhanced fluorescence signal as early as five hours after induction for production, suggesting that the developed biosensor can be used for screening fatty acid-producing strains at an early stage (FIG. 8). Overall, our results indicated that the developed fatty acid biosensors can sense both exogenous and endogenous fatty acids. They can be used for the detection of fatty acid in solutions, for high throughput screening of fatty acid-producing strains, and more importantly, have the potential to regulate metabolic pathways.

### Design of Fatty Acid-Regulatory Promoters

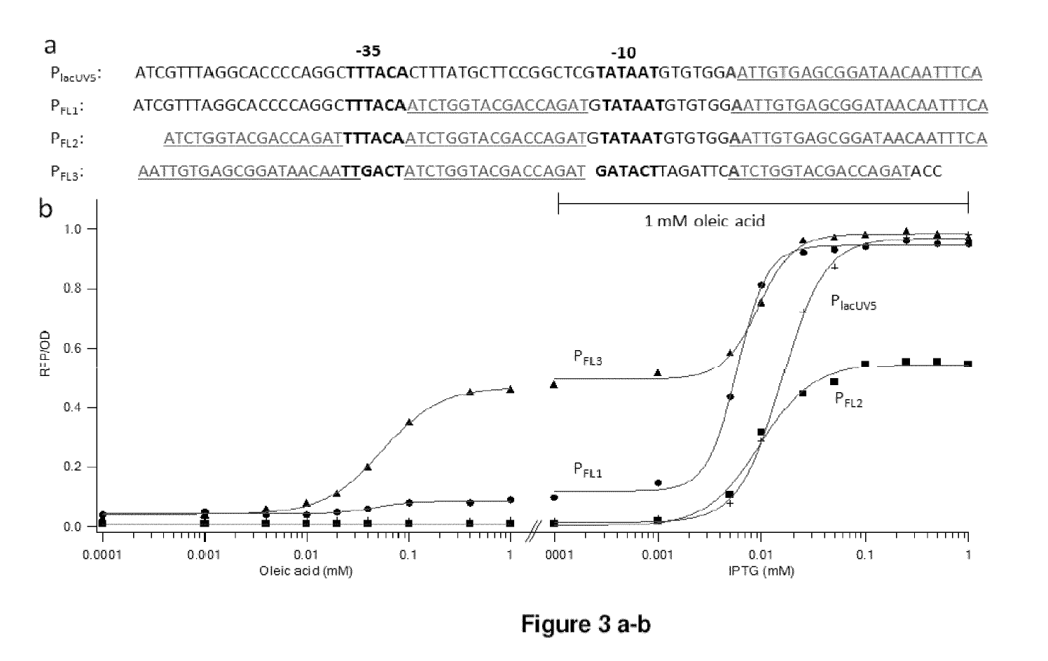

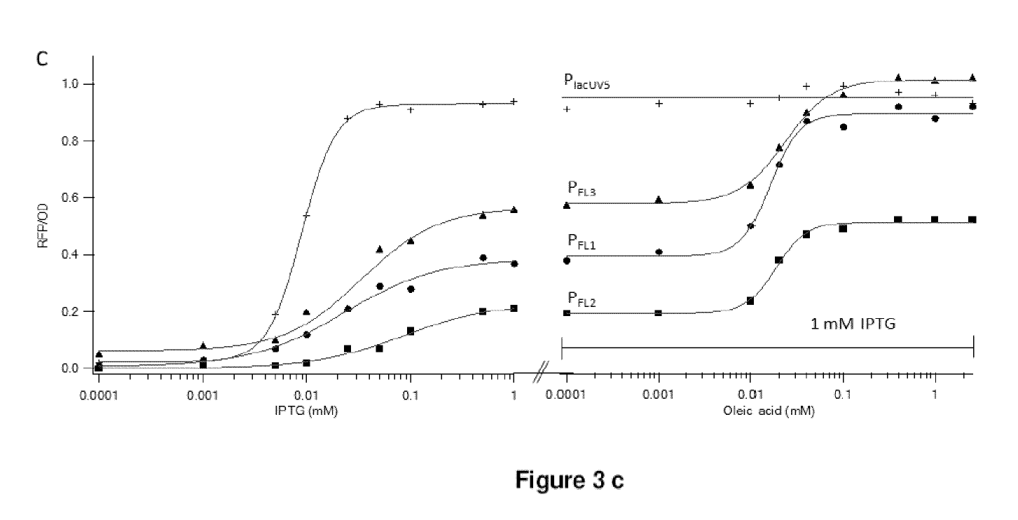

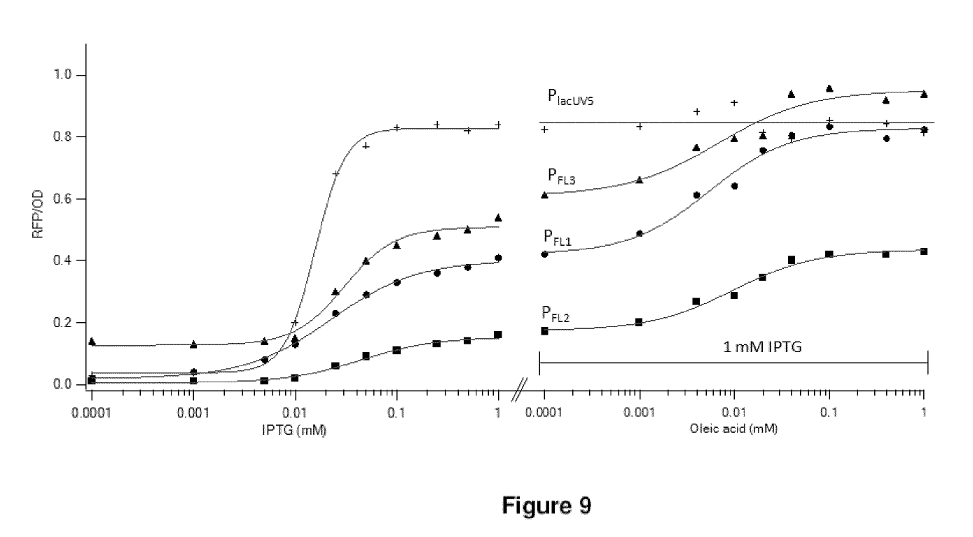

In order to use fatty acid biosensors to control engineered pathways for FAEE biosynthesis, it is essential to prevent leaky expression before induction for production. To do so, three hybrid promoters, P_{FL1}, P_{FL2}, and P_{FL3}, were created by combining the sequence of the IPTG inducible P_{lacUV5} with P_{LR} or P_{AR} (FIG. 3a). The hybrid promoters are expected to be fully activated by the presence of both fatty acid and IPTG. When they were analyzed at various inducer concentrations, in the case of P_{FL1} and P_{FL2}, where lacI-binding site was inserted downstream of the transcription start site, RFP expression was well repressed in the absence of IPTG. As contrast, P_{FL3} (FIG. 3b) was created by the insertion of lacI-binding site upstream of the −35 region. In the absence of IPTG, P_{FL3} behaved similarly with P_{AR}, exhibiting oleic acid dependent activation. This observation is consistent with previous studies that repression of a promoter at upstream region is less sensitive than the downstream region or the spacer region between −10 and −35²⁰. Titration of P_{FL3} with IPTG in the presence of 1 mM oleic acid continued to activate P_{FL3}. When the titration order was switched (IPTG followed by oleic acid), dual induction was observed for all the promoters (FIG. 3c). The designed promoters behaved robustly as changing copy numbers of promoters or repressor genes had litter effect on their behavior (FIG. 9).

### Sensory-Regulation for Biodiesel Production

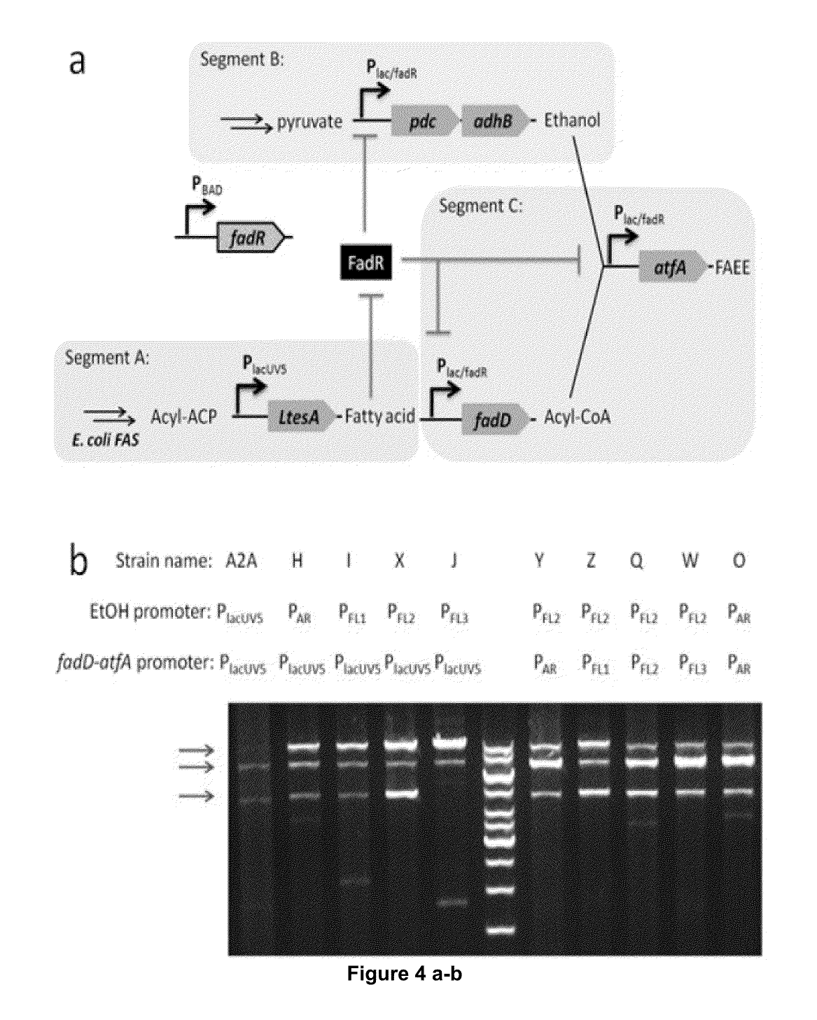

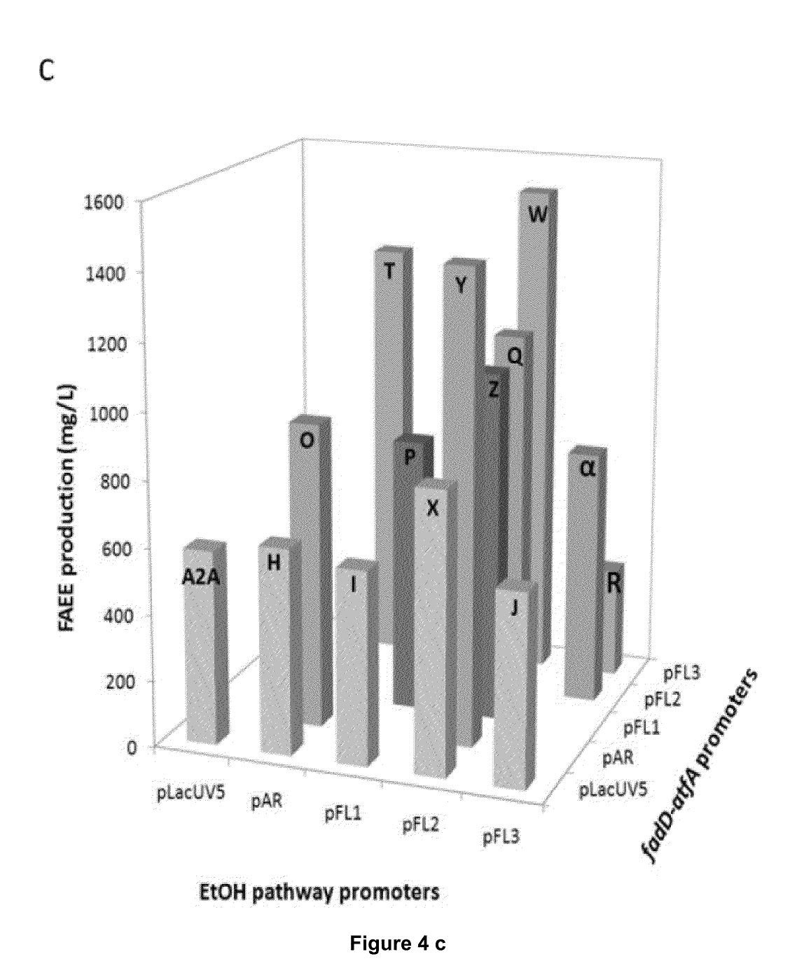

The fatty acid-regulatory promoters were applied to FAEE biosynthetic pathway to sense the availability of fatty acid and synchronize the biosynthesis of ethanol and acyl-CoA. To do so, fatty acid-regulatory promoters were cloned to control the expression of fadD, ethanol biosynthetic pathway, and atfA (FIG. 4a). At low fatty acid concentration, FadR repress the expression of the downstream pathways to prevent accumulation of toxic ethanol and unnecessary acyl-CoA. When there is sufficient fatty acid available, downstream pathways are expected to turn on simultaneously, which synthesize ethanol and acyl-CoA, and convert them into FAEE immediately. By changing the combination of promoters, 13 FAEE producing strains were first created (Table 1).

TABLE 1
List of FAEE production strains engineered in this study and compared
to the previous engineered A2A strain (Steen, E. J. et al. Microbial
production of fatty-acid-derived fuels and chemicals from plant biomass.
Nature 463, 559-562 (2010); hereby incorporated by reference).

| | | | | FAEE | Fatty acid |
|---|---|---|---|---|---|
| Strain | promoters | | | production | production |
| name | module A | module B | module C | (mg/L) | (mg/L |
| A2A^{a} | P_{lacUV5} | Pl_{acUV5} | P_{lacUV5} | 427 ± 38 | 49 ± 28 |
| H | P_{lacUV5} | P_{AR} | P_{lacUV5} | 734 ± 158 | 287 ± 77 |
| I | P_{lacUV5} | P_{FL1} | P_{lacUV5} | 600 ± 21 | 284 ± 68 |
| X | P_{lacUV5} | P_{FL2} | P_{lacUV5} | 879 ± 100 | 1054 ± 190 |
| J | P_{lacUV5} | P_{FL3} | P_{lacUV5} | 663 ± 117 | 267 ± 111 |
| O | P_{lacUV5} | P_{AR} | P_{AR} | 1044 ± 40 | 99 ± 1 |
| F1 | P_{lacUV5} | P_{FL1} | P_{AR} | 713 ± 27 | 74 ± 3 |
| Y | P_{lacUV5} | P_{FL2} | P_{AR} | 1463 ± 150 | 162 ± 93 |
| F2 | P_{lacUV5} | P_{FL3} | P_{AR} | 615 ± 23 | 70 ± 3 |
| F3 | P_{lacUV5} | P_{AR} | P_{FL1} | 971 ± 30 | 153 ± 4 |
| P | P_{lacUV5} | P_{FL1} | P_{FL1} | 910 ± 129 | 222 ± 3 |
| Z | P_{lacUV5} | P_{FL2} | P_{FL1} | 1055 ± 140 | 603 ± 220 |
| F4 | P_{lacUV5} | P_{FL3} | P_{FL1} | 825 ± 7 | 64 ± 10 |
| F5 | P_{lacUV5} | P_{AR} | P_{FL2} | 1067 ± 14 | 139 ± 4 |
| F6 | P_{lacUV5} | P_{FL1} | P_{FL2} | 427 ± 6 | 67 ± 4 |
| Q | P_{lacUV5} | P_{FL2} | P_{FL2} | 1021 ± 112 | 263 ± 56 |
| α | P_{lacUV5} | P_{FL3} | P_{FL2} | 771 ± 232 | 878 ± 55 |
| T | P_{lacUV5} | P_{AR} | P_{FL3} | 1289 ± 106 | 171 ± 61 |
| V | P_{lacUV5} | P_{FL1} | P_{FL3} | 1157 ± 175 | 400 ± 195 |
| W | P_{lacUV5} | P_{FL2} | P_{FL3} | 1503 ± 87 | 128 ± 11 |
| R | P_{lacUV5} | P_{FL3} | P_{FL3} | 333 ± 81 | 1149 ± 45 |
| C1 | P_{lacUV5} | P_{C1} | P_{FL3} | 759 ± 238 | 1227 ± 114 |
| C2 | P_{lacUV5} | P_{C2} | P_{FL3} | 751 ± 211 | 354 ± 20 |
| C3 | P_{lacUV5} | P_{C3} | P_{FL3} | 601 ± 197 | 764 ± 61 |
| C4 | P_{lacUV5} | P_{C4} | P_{FL3} | 662 ± 198 | 850 ± 54 |
| C5 | P_{lacUV5} | P_{C5} | P_{FL3} | 745 ± 126 | 652 ± 3 |
| C6 | P_{lacUV5} | P_{C6} | P_{FL3} | 780 ± 92 | 638 ± 7 |

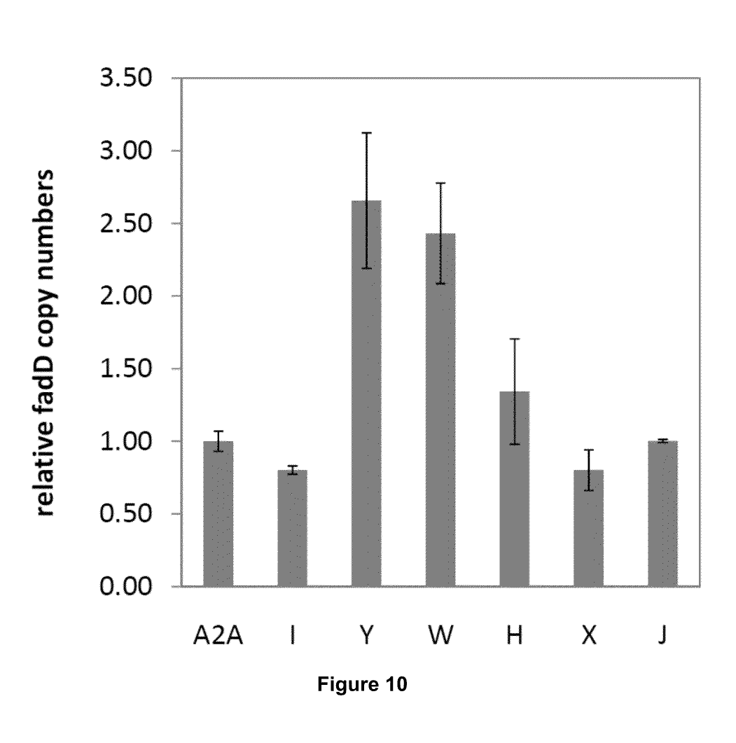

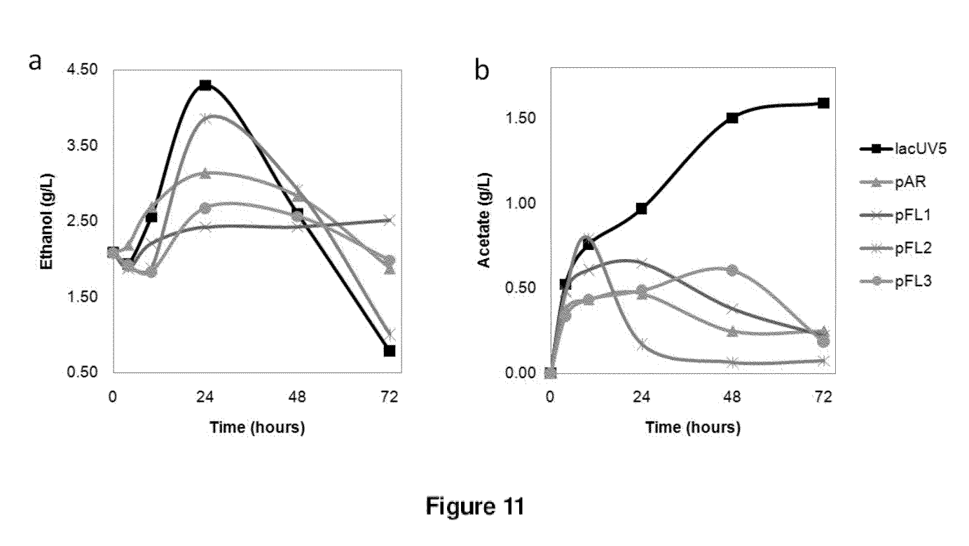

Our unpublished results suggested that genes in heterologous pathways were not stable during FAEE production, presumably due to the accumulation of toxic intermediates and proteins. We characterized the gene stability of FAEE-producing strains by isolating the plasmid DNAs after FAEE production. As compared to A2A, strains using fatty acid-regulatory promoters had higher plasmid integrity and proper copy number ratios as shown by gel electrophoresis (FIG. 4b). The amount of fadD gene was further quantified by qPCR and compared. Consistent with results from gel electrophoresis, strains using fatty acid-regulatory promoters maintained higher copy of this gene (FIG. 10), indicating that fatty acid-regulatory promoters were able to improve gene stability.

Next, FAEE production yields were measured. Most of strains using fatty acid-regulatory promoters had enhanced production yields (FIG. 4c). Among them, two strains, Y and W, which contain P_{FL2} controlling the expression of genes in segment B (ethanol pathway) and P_{AR} or P_{FL3} controlling genes in segment C (fadD-atfA), had the highest yields. They increased FAEE production by three fold as compared with the previous engineered A2A strain, reaching 1.5 g/L after three days' incubation, corresponding to 28% of the theoretical limit.

### Static Regulation Versus Dynamic Regulation

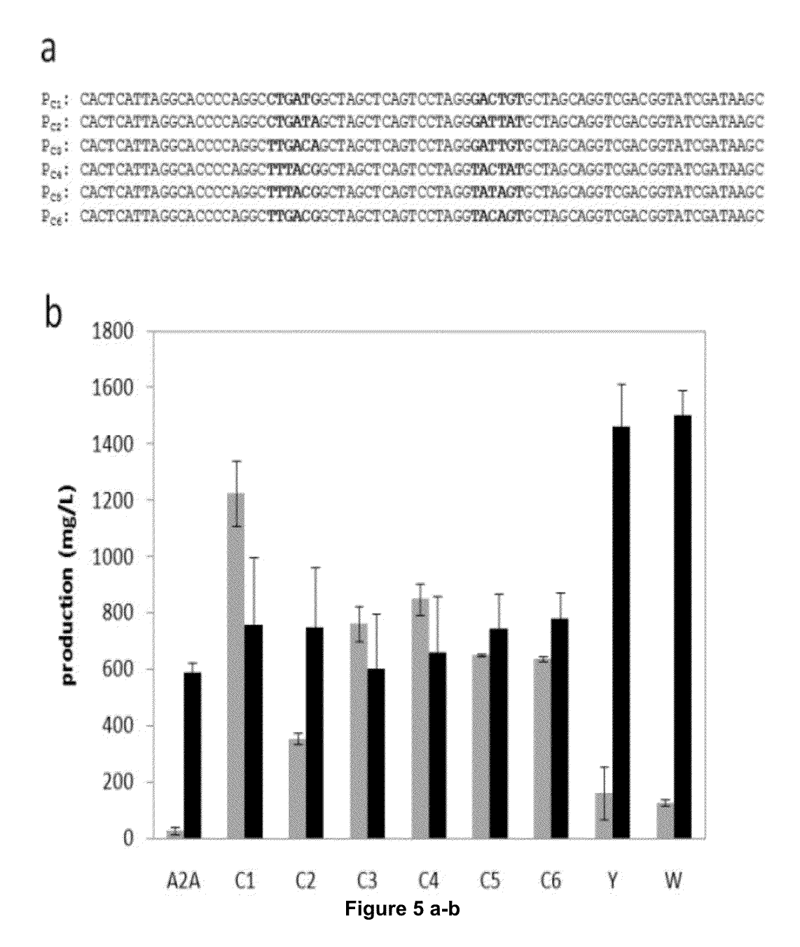

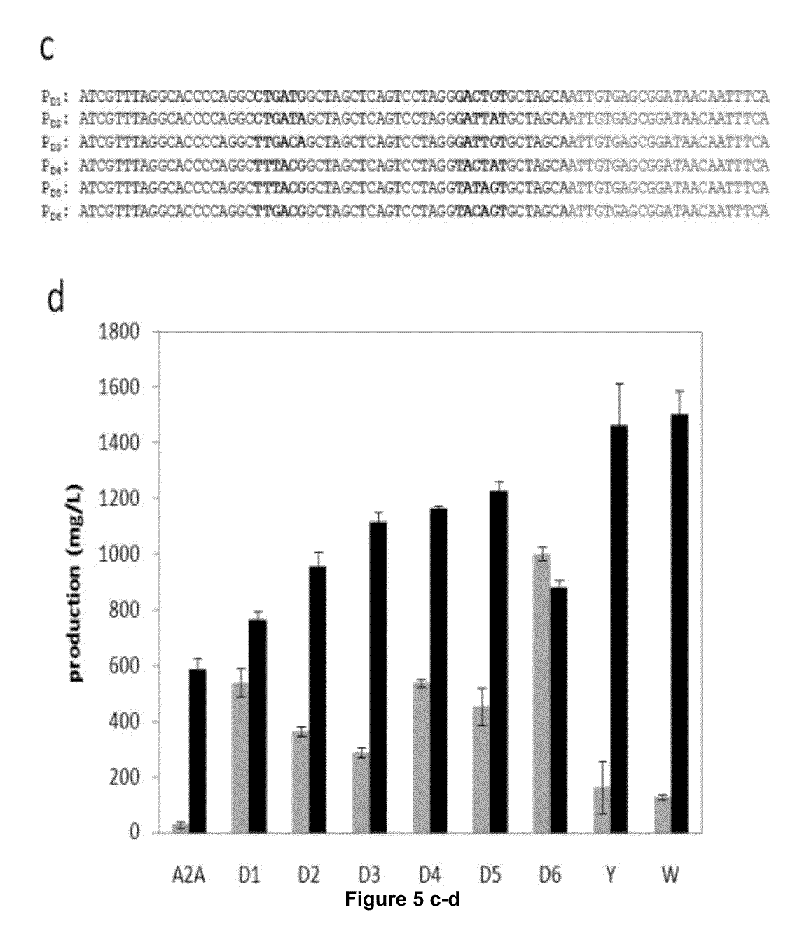

In order to confirm that the yields were enhanced because of the dynamic regulation created by the sensory-regulation system rather than simply change of promoter strength, we used a series of static promoters to control the same pathway and compared their effects in FAEE production. Six constitutive promoters (P_{C1} to P_{C6}, FIG. 5a) from the MIT registry were first chosen to create static regulation on ethanol biosynthesis. P_{C1} to P_{C6} have varied sequences at the −10 and −35 regions and were previously characterized to cover a wide range of strength from weak to strong. They were cloned to substitute the P_{FL2} in segment B of the best FAEE producing strain W (Table 1). The resulting strains, C1 to C6, only produced half amount of FAEE as compared with W strain. Instead, large amounts of free fatty acids were accumulated in C1 to C6 (FIG. 5b), suggesting the imbalance of metabolism. To further prove that dynamic regulation on acyl-CoA synthesis is also important, promoters in segment C of Y and W strains were substituted to a series of static promoters (Table 1). This time, P_{C1} to P_{C6} were modified by integration with the lacI-binding sequence to prevent expression before induction, which generated a series of inducible promoters (P_{D1} to P_{D6}, FIG. 5c) with different strengths. When the promoter strength was increased from P_{D1} to P_{D6}, FAEE production yield was first increased then decreased. Nevertheless, all the strains using static regulation accumulated more fatty acids and their FAEE production yields were lower than Y and W. Taken together, our results have shown that dynamic regulation of either ethanol synthesis or fatty acid activation to acyl-CoA enhanced production yield. To have optimal FAEE production, it is important to synchronize these two pathways according to the availability of cellular fatty acid.

### REFERENCES CITED

1. Ajikumar, P. K. et al. Isoprenoid pathway optimization for Taxol precursor overproduction in Escherichia coli. Science 330, 70-74 (2010).
2. Ro, D. K. et al. Production of the antimalarial drug precursor artemisinic acid in engineered yeast. Nature 440, 940-943 (2006).
3. Schmidt-Dannert, C., Umeno, D. & Arnold, F. H. Molecular breeding of carotenoid biosynthetic pathways. Nat Biotechnol 18, 750-753 (2000).
4. Steen, E. J. et al. Microbial production of fatty-acid-derived fuels and chemicals from plant biomass. Nature 463, 559-562 (2010).
5. Atsumi, S., Hanai, T. & Liao, J. C. Non-fermentative pathways for synthesis of branched-chain higher alcohols as biofuels. Nature 451, 86-89 (2008).
6. Harcum, S. W. & Bentley, W. E. Heat-shock and stringent responses have overlapping protease activity in Escherichia coli. Implications for heterologous protein yield. Appl Biochem Biotechnol 80, 23-37 (1999).
7. De Mey, M., Maertens, J., Lequeux, G. J., Soetaert, W. K. & Vandamme, E. J. Construction and model-based analysis of a promoter library for E. coli: an indispensable tool for metabolic engineering. BMC Biotechnol 7, 34 (2007).
8. Pfleger, B. F., Pitera, D. J., Smolke, C. D. & Keasling, J. D. Combinatorial engineering of intergenic regions in operons tunes expression of multiple genes. Nat Biotechnol 24, 1027-1032 (2006).
9. Salis, H. M., Mirsky, E. A. & Voigt, C. A. Automated design of synthetic ribosome binding sites to control protein expression. Nat Biotechnol 27, 946-950 (2009).
10. Farmer, W. R. & Liao, J. C. Improving lycopene production in Escherichia coli by engineering metabolic control. Nat Biotechnol 18, 533-537 (2000).
11. Zhang, F. & Keasling, J. D. Biosensors and their applications in microbial metabolic engineering. Trends Microbiol (2011).
12. Zhang, F., Rodriguez, S. & Keasling, J. D. Metabolic engineering of microbial pathways for advanced biofuels production. Curr Opin Biotechnol (2011).
13. Fujita, Y., Matsuoka, H. & Hirooka, K. Regulation of fatty acid metabolism in bacteria. Mol Microbiol 66, 829-839 (2007).
14. Cronan, J. E., Jr. In vivo evidence that acyl coenzyme A regulates DNA binding by the Escherichia coli FadR global transcription factor. J Bacteriol 179, 1819-1823 (1997).
15. DiRusso, C. C., Heimert, T. L. & Metzger, A. K. Characterization of FadR, a global transcriptional regulator of fatty acid metabolism in Escherichia coli. Interaction with the fadB promoter is prevented by long chain fatty acyl coenzyme A. J Biol Chem 267, 8685-8691 (1992).
16. Iram, S. H. & Cronan, J. E. Unexpected functional diversity among FadR fatty acid transcriptional regulatory proteins. J Biol Chem 280, 32148-32156 (2005).
17. Henry, M. F. & Cronan, J. E., Jr. A new mechanism of transcriptional regulation: release of an activator triggered by small molecule binding. Cell 70, 671-679 (1992).
18. Lutz, R. & Bujard, H. Independent and tight regulation of transcriptional units in Escherichia coli via the LacR/O, the TetR/O and AraC/I1-12 regulatory elements. Nucleic Acids Res 25, 1203-1210 (1997).
19. van Aalten, D. M., DiRusso, C. C. & Knudsen, J. The structural basis of acyl coenzyme A-dependent regulation of the transcription factor FadR. EMBO J 20, 2041-2050 (2001).
20. Lanzer, M. & Bujard, H. Promoters largely determine the efficiency of repressor action. Proc Natl Acad Sci USA 85, 8973-8977 (1988).

Each of the reference cited is hereby incorporated by reference as though each is individually and separately incorporated by reference.

### Example 3

### Fatty Acid-Responsive Biosensors

A series of fatty acid-responsive promoters are engineered by the insertion of fadR-binding DNA sequences inteo several phage promoters. Biosensor plasmids are created by using the fatty-acid promoters to control the expression of RFP. The fadR gene is cloned into another plasmid, pE8a-fadR, under the control of a pBAD promoter. Plasmid pE8a-fadR is then cotransformed together with one of the biosensor plasmids into E. coli to create a fatty acid sensing strain.

Fatty acid biosensors are tested by adding oleic acid (C18;1) into the growth media. Cell culture fluorescence intensity is measured and normalized to OD after 12 hours. All of the edesigned biosensors exhibited fatty acid concentration-dependent fluorescence. Particularly, when the plasmid pBARk-RFP is used, more than 50-fold increase in fluorescence signal is observed over a broad range of oleic acid concentration. When pNARk-RFP and pE8a-fadR is transformed into a fatty acid-producing strain (ΔfadE:DH1 E. coli strain with pA5c-tesA), the strain exhibited 20-fold higher fluorescent signal than a non-fatty acid-producing strain (DH1 E. coli straion), indicating the biosensor can be used to detectinternally produced fatty acid. These results also indicate that the engineered biosensors can be used for high throughput screening to select for fatty acid-producing strains.

Fatty acid sensors are also used to create dynamic regulation for FAEE production. Fatty acid-responsive prmoters are used tio control the expression of acyl-CoA synthase, wax-ester synthase and two genes leading to the production of ethanol from pyruvate (pdc and adhB). All of the engineered strains exhibit elevated FAEE production levels. Strains Y and W (Table 1) produced about 1.5 g/L after 72 hours incubation in test tubes, which is three times higher than the A2A strain.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A genetically modified host cell capable of producing fatty acid comprising an increased expression of FadR, or a functional variant thereof.

2. The genetically modified host cell of claim 1, wherein the host cell comprises more than one copy of a gene encoding FadR, or a functional variant thereof.

3. The genetically modified host cell of claim 2, wherein at least one copy of the gene is operably linked to an inducible or constitutive promoter.

4. The genetically modified host cell of claim 1, wherein the FadR, or a functional variant thereof, comprises an amino acid sequence that is at least 70% identical to the amino acid sequence of SEQ ID NO:1.

5. The genetically modified host cell of claim 1, wherein the host cell further comprises one or more enzymes, or a functional variant thereof, capable of capable of producing a fatty acid alkyl ester (FAAE) from a fatty acid and an alkyl alcohol.

6. The genetically modified host cell of claim 5, wherein the FAAE is a fatty acid ethyl ester (FAEE) and the alkyl alcohol is ethanol.

7. The genetically modified host cell of claim 1, wherein the host cell is a microorganism from the Escherichia, Salmonella, Vibrio, Pasteurella, Haemophilus, or Pseudomonas genus.

8. A method of producing a fatty acid or fatty acid alkyl ester (FAAE) in a genetically modified host cell, comprising: culturing the genetically modified host cell of claim 1 in a medium under a suitable condition such that the culturing results in the genetically modified host cell producing the fatty acid or FAAE, and optionally recovering the fatty acid or FAAE from the medium, wherein the recovering step is concurrent or subsequent to the culturing step.

9. The method of claim 8, wherein the FadR, or a functional variant thereof, comprises an amino acid sequence that is at least 70% identical to the amino acid sequence of SEQ ID NO:1.

10. The method of claim 8, wherein the host cell further comprises one or more enzymes, or a functional variant thereof, capable of capable of producing a fatty acid alkyl ester (FAAE) from a fatty acid and an alkyl alcohol.

11. The method of claim 10, wherein the FAAE is a fatty acid ethyl ester (FAEE) and the alkyl alcohol is ethanol.

12. The method of claim 8, wherein the host cell is a microorganism from the Escherichia, Salmonella, Vibrio, Pasteurella, Haemophilus, or Pseudomonas genus.

13. A genetically modified host cell comprising a fatty acid biosensor and one or more fatty acid-responsive promoter operably linked to one or more genes of interest that is heterologous to the fatty acid-responsive promoter, wherein the expression of the fatty acid biosensor in the host cell is increased compared to the host cell if not unmodified.

14. The genetically modified host cell of claim 13, wherein the host cell is a microorganism from the Escherichia, Salmonella, Vibrio, Pasteurella, Haemophilus, or Pseudomonas genus.

15. A genetically modified host cell comprising a fatty acid-responsive transcription factor, and a fatty acid-responsive promoter operatively linked to a reporter gene, wherein the fatty acid-responsive promoter is capable of expression of the reporter gene with an activated form of the fatty acid-responsive transcription factor.

16. The genetically modified host cell of claim 15, wherein the reporter gene confers a positive selection on the host cell under a certain growth condition.

17. The genetically modified host cell of claim 16, wherein the reporter gene is an antibiotic resistance gene.

18. The genetically modified host cell of claim 15, wherein the reporter gene confers a negative selection on the host cell under a certain growth condition.

19. A method for sensing acyl-CoA and/or one or more fatty acids, comprising: (a) providing the genetically modified host cell of claim 15, and (b) detecting the expression of the reporter gene.

20. A method for screening or selecting a host cell that produces an acyl-CoA and/or one or more fatty acids, comprising: (a) providing the modified host cell of claim 15, (b) culturing the host cell, and (c) screening or selecting the host cell based the expression of the reporter gene by the host cell.
